(19) United States
(12) Patent Application Publication
(10) Pub. No.: US 2007/0003512 A1
(43) Pub. Date: Jan. 4, 2007

(54) Bisphosphonate resinates

(76) Inventors: Richard F. Stockel, Bridgewater, NJ (US); Edward G. Budnick, Flemington, NJ (US)

Correspondence Address: RICHARD F STOCKEL, 475 ROLLING HILLS ROAD, BRIDGEWATER, NJ, 08807, US

(21) Appl. No.: 11/156,513

(22) Filed: Jun. 20, 2005

(51) Int. Cl. A61K 31/675; A61K 31/66

(52) U.S. Cl. 424/76.1; 514/89; 514/102

(57) ABSTRACT

The present invention is directed to novel and new oral parenteral compositions comprising various bisphosphonates in conjunction with bound or unbound ion exchange resins, or mixtures of the two types of resins thereof. Accordingly, the said dosage forms effect the delivery to the lower intestinal tract in humans or animals prohibiting the exposure of the bisphosphonates to the epithelial and mucosal tissues of the buccal cavity, pharynx, esophagus, and stomach. These drug-resinates also have the systemic effect of providing a slow release mechanism, thereby extending the efficacy of the treatment for a longer period of time. The drug resinates can also incorporate ionic charged bioactive molecules containing carboxylate, amino or positively charged amino groups having synergistic value in conjunction with bisphosphonates.

### INTRODUCTION

This invention deals with new and novel compositions and formulations of bisphosphonates with cation or anion ion exchange resins depending on the chemical structure of the bisphosphonate. The resulting drug-resinate which can be chemically and/or physically bound together, results in lessening side effects and provides longer therapy by extending the treatment by virtue of a slow release mechanism.

Bisphosphonates are a family of drugs that have been used in the management of disorders of calcium and bone metabolism for the last three decades. The therapeutic potential of bisphosphonates is due to their potent inhibition of osteoclast-mediated bone resorption. By varying the substituents on the methylene group on the bisphosphonates many different derivatives have been synthesized and several have been commercialized.

Bisphosphonates all have two phosphonic acid functionalities, the rest of the molecule can have functional groups which are neutral hydroxyl, alkyl, arylate basic, e.g., amine, heterocyclic acid, e.g., carboxylic acid, sulfonic acid.

In the case of bisphosphonates having only neutral groups, both weak or strong anionic ion exchange resins can be used.

In the case of bisphosphonates having basic groups, all four types of ion exchange resins can be utilized, e.g., weak or strong cation, or weak and strong anionic exchange resins are feasible depending on the pH of the medium and the pKa/pKb of the exchanger and the drug.

When bisphosphonates have only acidic groups, then only anionic exchange resins can be used.

Of course, it is well known in the field of ion exchange theory, that the pH, medium ionic strength, ion capacity and other factors can influence the release rate thereby affecting the treatment. Therefore, optimization is required to obtain the best release rate.

The teachings of this invention are applicable to any bioactive bisphosphonate, because they can all be bound or exchanged with specific types of ion exchange resins. Longer delivery rates can be obtained by coating the resinate (bisphosphonate-ion exchange resin) with water-soluble or hydrocolloid polymers.

Said dosage forms prohibit the exposure of the bisphosphonates to the epithelial and mucosal tissue of the buccal cavity, pharynx, esophagus, and the stomach and thereby protects said tissues from erosion, ulceration or other like irritation. Accordingly, the bisphosphonate-resinates dosage forms effect the delivery to the lower intestinal tract of said human or other mammal of a safe and effective amount of the bisphosphonate drugs, and substantially alleviate the esophagitis or esophageal irritation which offend accompanies the oral administration of bisphosphonates active ingredients.

Resinate is the term used to describe the complex formed between a drug and an ion exchange resin. The term is appropriate because the complexation mechanium is salt formation and the resinate can be considered as a salt form of the bisphosphonate in which the counterion is a polymeric ion. The term resination is used to describe the process of making a resinate.

Biphosphonate-resinates are easily prepared when the drug molecule has ionic functionality present. Specifically, carboxylic, amine, phosphonic moieties will bind to a ion exchange resin. All bisphosphonates have one or more of these functions, therefore the resinate can simply be prepared.

Due to the binding capacity of ion exchange resins, the drug molecular weight should be ≦1,000, otherwise the tablet would be unacceptability large or an unpalatable liquid formulation would be necessary to drink.

Fortunately, all commercially bisphosphonates meet this criteria.

Ion exchange resins have been used for many years in pharmaceutical formulations. Their uses have ranged from simple excipients for tablet disintegration, to the rate controlling function in extended release formulations.

The resins used to prepare bisphosphonate-resinates of this invention are totally insoluble in all solvents and at all pH's, combined with their particle size means that they are not absorbed by the body, and so have proven to be non-toxic and very safe

An example (not all inclusive, but merely illustrative) between alendronate and a anion exchange can be depicted as

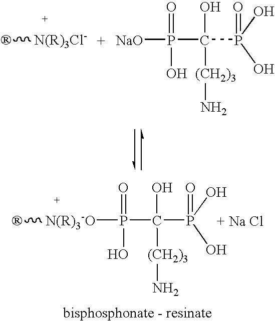

Other advantages of the compositions of this invention, bisphosphonates—ion exchange resin, are taste masking because the resinate is insoluble and the resinate passes practically unchanged through the gastric system into the intestinal tract. Another desirable characteristic of the resinate is the achieve extended release.

With respect to the drug release rates, fortunately there are several modifications which can be utilized to extent the rates to assure better bioavailability beyond the resinate formation.

One such simple modification is to coat the resinate with a slow aqueous dissolving water soluble or hydrocolloid polymeric substance, e.g., cellulose, hydroxyalkylcellulose, corn starch, gum Arabic, alginate, polyvinyl alcohol, carboxymethylcellulose, polyvinyl pyrollidione, polyethylene glycol 4000, carrageenan. These specific examples are just a few, which can function as coatings for the bisphosphonates-resinates of this invention. Many other polymeric compositions which are well known to skilled chemists can be used as the coating material thus the above examples are not all inclusive.

A second approach to extent the release of this invention is achieved by co-administering a bisphosphonate-resinate and an unloaded resin. In general the weight ratio of resinate to unloaded resin was 1:1.1 to 1:2 w/w. This yields an essentially constant rate release over time. By changing variables such as loading, particle size and ratio it is possible to optimize the release of bisphosphonates.

The mechanism for this effect can be explained as follows: in the GI tract the resinate starts to release bisphosphonate as it establishes thermodynamic equilibrium. At the same time the unloaded resin absorbs some of the bisphosphonate, while some of the drug is also absorbed by the body. On the other hand the unloaded resin starts to absorb some drug molecules to establish its equilibrium. This latter reaction contributes to the extended time period of release of the drug. Obviously, both the loaded and unloaded forms can be coated with an appropriate polymer as described previously.

Yet another option for controlled release can be used for this invention, which involves the co-administration of the drug and unloaded resin. These can be formulated with or without an appropriate coating, depending on the desired release rate. The mechanism is similar to the previous methods since the affinity of the drug for the unloaded resin is high so that the resinate quickly forms in the body medium and subsequently is gradually released.

The bisphosphates—resonates of this invention are preferably those for internal administration, e.g., unit dosage forms such as oral, vaginal and rectal formulations, e.g., tablets, capsules, syrups, drops or suppositories. It is also possible to use stable slurries of these formulations that are used for injections.

The novel pharmaceutical compositions of the present invention are prepared in a manner known per se, for example by conventional mixing, granulating, confectioning, dissolving or lyophilizing method.

Suitable carriers are in particular fillers such as sugar, for example lactose, saccharose, mannitol, or sorbitol, cellulose or derivatives thereof and/or calcium phosphates and also binders such as starches and/or water-soluble or hydrocolloids. The latter can also be used as an outer coating to achieve various degrees of control release. Other adjuncts useful as glidants and lubricants like silica, talc, stearic acid/derivatives thereof, are also used for the formulation of these drugs.

Gelatin capsules are yet another form of delivery. Plasticizers like glycerol, propylene glycol, low molecular weight polyethylene glycols or sorbitol are useful in the preparation of said capsules.

Ion exchange applications may be carried out by either column or batch techniques. Column operations are usually preferable, although the choice of method depends entirely upon the application in question.

In column operations the ion exchange resin is placed in a vertical column to form a bed. The solution to be treated flows through this column until a target end-point is reached.

In batch operations the ion exchange resin is simply agitated in a vessel together with a given quantity of the solution to be treated. When the target end-point is reached, the treated solution is removed by decanting, and the resin maybe regenerated.

Usually quantitative exchange is much easier to achieve by column technique. An ion exchange column may be regarded as a series of equilibrium systems, much like the theoretical plates of distillation theory. As the solution proceeds down the column, the exchange reaction is continuously driven in the forward direction by the equilibrium requirement of each new theoretical plate encountered.

There are four fundamental types of synthetic ion exchange resins available. Each type is analogous to common acids or bases and undergoes similar reactions. These are:
Strongly acidic cationic exchange resins analogous to sulfuric acid
Weakly acidic cation exchange resine analogous to carboxylic acids
Strongly basic anion exchange resin analogous to sodium hydroxide
Weakly basic anion exchange resin analogous to ammonium hydroxide

Weakly acidic and weakly basic exchangers do not react with salts, nor do they react with weak bases or acids respectively, while strongly acid and strongly base exchangers do react in both cases.

In selecting the proper ion exchange resin to prepare the resonates of this invention there are several characteristics of the resins which need to be considered. The proper ionic functionality must be chosen. Therefore, when preparing the resinate of a sodium or other water-soluble salt of a bisphosphonate, a strongly basic anion exchange resin is preferred. If the bisphosphonate is entirely in the acid form then a strongly and/or weakly basic anion exchange resin can be utilized.

Ion exchange resins can be prepared by various techniques to produce either micro reticular or macro reticular porosity. Micro porous resins are essentially homogeneous crosslinked gels where the pore structure is the distance between polymeric chains. Macro porous resins contain a significant nongel porosity in addition to the normal gel porosity. The gel resin is a continuous polymeric phase while the macro reticular resins are clearly shown to be agglomerates of randomly packed micro spheres with a continuous nongel pore structure. The exchangers of the gel type porosity are the preferred resins for the preparation of the bisphosphonates resonates of this invention. The gel type exchangers (micro porous) are usually sold with 1 to about 8 wt. % of a crosslinking agent to insolubilize the resin.

Preferred ion exchange resins taught in this invention are small particle sized, usually from 200 to 400 mesh. Most preferably about 400 mesh or less than 45 microns. This allows the drug-resin (resinate) to be formed into a tablet, gel, and slurry more easily for administration to a patient.

Any bioactive bisphosphonate useful for the treatment of osteoporosis can be affixed to an appropriate resin to form the resonates by an ion exchange mechanism. The resulting resinate can also be coated with of water-soluble or hydrocolloid polymeric material to lengthen the release rate. Suitable bisphosphonates are pamidromate, ibandronic acid, ibandronate, resedronate, cimadronate, clodronate, etidronic acid, neridronate, olpadronate, piridronate, tiludronate, zolendronate, icadronate, and pharmaceutically acceptable salts thereof.

A unique aspect of our invention involves the anionic exchange capability of the resin, whereby certain non-steroidal anti-inflammatory drugs or NSAID's, which are able to form an anion can be administered along with a bisphosphonate simultaneously.

Examples of some, not all inclusive, NSAID's are aspirin, ibuprofren, didofenal, diflunisal, etodolac, fenoprofen, flurbisprofen, ibuprofen, indomethacin, ketoprofen, ketofolac, mefenamic acid, naproxen, oxaprozin, piroxicam, salsalate, sulindac, tolmetin and the like.

Other bioactive molecules, capable of forming an exchangeable anion with the resin anion, are vitamins C and E, thereby resulting in synergy. Both vitamins can be exchanged with an anionic exchange resin. Vitamins C and E are known to benefit the maintenance of healthy bone structure and growth. For example vitamin C functions to help maintain collagen, a protein necessary for forming skin, ligaments, teeth, and bones. Vitamin E derivatives have beneficial effects on bone calcium in adrenalectomized animal studies (S. Ima-Nerwana, J. Med. Food, 2004, Spring 7(1) 45-51).

It is also known that patients with peptic disorders, e.g., peptic ulcers generally have low leucocyte levels of ascorbic acid. This is also true for patients with gastro duodenal problems. Thus the availability of vitamin C would help to alleviate these conditions.

Amino carboxylic and amino phosphonic chelating agents like EDTA can also be incorporated as an anion.

Obviously several biological activities could be administered using the teachings of this invention, provided they have a therapeutic value.

Commercially available ion exchange resins are sold by several chemical companies including Dow Chemical Company and Rohm & Haas Company, to mention a few. Some specific commercial exchangers for the purpose of forming resonates with bisphosphonates for this invention are Rohm & Haas, Duolite AP 143 and Dow's Dowex 1×2, Dowex 1×4 and Dowex 1×8. These are anionic ion exchange resins having from 1 to 8% wt. of a crossing-linking network, small particle size from 200-400 mesh, sufficient binding capacity, and constitute a gel like resin (micro reticular), rather than a micro reticular structure. While macro resins are functional micro resins are preferred. Anyone skilled in the art of ion exchange resins could substitute other chemically different resins than those described. The concept of a resinate is essentially the same no matter which ion exchange resin is chosen.

### EXPERIMENTAL

### Preparation of Resinates

Process to prepare a resinate with sodium alendronate (molecular weight 325.12) and Dowex 1×200-400 mish (binding capacity 3.5 megv./1 g dry weight) was carried out by adding to 250 mls of distilled water, 3.803 grams of alendronate sodium trihydrate (mw 325.12) and about 10 grams of the exchanger. This resulted in near full binding of the drug to the anionic ion exchanger. Slurry the components at r.t. for 24-48 hours followed by filtration and washing with distilled water, then dried. Nitrogen elemental analysis confirmed the presence of the bisphosphonate.

Partial Binding

Various levels of drug (bisphosphonates) resin can be prepared by the same procedure as described above by reducing the concentration of the drug. This will result in a system whereby the resin is in excess and consequential the unbound resin will compete with the GI tract to accept free drug molecules. The result is a further reduction in the release rate with an essentially constant rate over time. Generally the ratio of resinate to unloaded resin was 1:1.5 w/w.

Admixture

The method involves the co-formulation with the drug and resin with no pre-binding. Due to the strong electrostatic binding of a salt of a bisphosphonate and an ion exchange resin, e.g., strong anionic type will occur in the GI-tract. Generally the ratio of resin to drug is 1.5 to 2.0:1.0 w/w.

A preferred dosage form to achieve a longer release rate is a matrix tablet containing, an active ingredient (bisphosphonate), Methocel®, fillers, lubricants and excipients. This type of tablet uses a water-soluble methocel as a rate controlling polymer and may be tableted by direct compression or conventional wet granulation. A hydrophilic matrix controlled release system is relatively easy to formulate as a robust dynamic system composed of polymer wetting, hydration and desolation.

Water permeates into the tablet, causing the gel layer to become thicker. The resinate or, resinate—resin or admixture of resin and drug diffuses out of the gel layer at a rate controlled by the gel viscosity. The primary release mechanism is by diffusion or erosion through the gel layer.

A preferred oral tablet weighs between 0.250 to 2.750 grains, with the bisphosphonate preferred range from 25 to 450 mg and a preferred range of Methocel® of 20 to 30 wt. percent of the total tablet weight. The remaining components of the tablet include fillers and lubricants.

### SOME EXAMPLES OF THE TABLET COMPOSITIONS ALL INVOLVING ALENDRONATE AS THE BISPHOSPHONATE (SINGLE TABLET)

| | | |
|---|---|---|
| 1. | Resinate-drug complex | 300 mg |
| | lactose | 100 mg |
| | corn starch | 10 mg |
| | PEG-6000 | 5 mg |
| | Magnesium stearate | 5 mg |
| 2. | Resinate-drug complex | 300 mg |
| | lactose | 35 mg |
| | colloidal silica | 5 mg |
| | microcrystalline cellulose | 20 mg |
| | corn starch | 30 mg |

The amount of bisphosphonate in both examples is about 80 mg.

Overall this invention advances the therapeutic. treatment of osteoporosis by disclosing a new control release system with biological bisphosphonates and optionally include other synergistic additives.

Advantages include:
Control release
Improved bioavailability
Reduced irritation
Mask bitter taste
Incorporate analgesic agents, anine narcotics, vitamins or pro-vitamins, hormones, and/or neutraceuticals.

While I have described certain preferred embodiments of my invention, many modifications there of may be made without departing from the spirit of the invention and I do not wish to be limited to the detailed specifications, examples, chemical compositions, or formulations herein set forth, but desire to avail myself of all changes within the scope of the appended claims.

We claim:

1. A pharmaceutical composition or admixture comprising a bioactive bisphosphonate drug and a ion exchange resin capable of binding to the drug by electrostatic forces either before administration of the drug, or in-vivo and optionally having an enteric coating consisting of a water-soluble or hydrocolloid polymer to lengthen the delivery rate.

2. The drug of claim 1, wherein the bioactive bisphosphonate is selected from the group consisting of pamidronate, minodronate, ibandronic, risedronate, cimadronate, clodronate, neridronate, olpadronate, piridronate, teludronate, zolendronate, icadronate, alendronate, or etidronate, has at least one water-soluble alkali or alkaline atom as a salt of a phosphonic acid.

3. The ion exchange resin of claim 1 wherein said exchanger is a strongly basic anion exchange resin, when the bisphosphonate has at least one neutralized phosphonic salt with a water soluble alkali or alkaline metal ion.

4. The drug of claim 1, wherein the bioactive bisphosphonate is selected from the group consisting of pamidronate, minodronate, ibandronic, risedronate, cimadronate, clodronate, neridronate, olpadronate, piridronate, teludronate, zolendronate, icadronate, alendronate or etidronate in the free acid form.

5. The ion exchange resin of claim 1 wherein said exchanger is a weak basic anion exchange resin, when the bisphosphonate is in the free acid form.

6. The resinate of claim 3 wherein the bisphosphonate is near fully bound to the exchanger.

7. The resinate of claim 3 wherein the resin and bisphosphonate has a 1.5 to 1.0 w/w ratio.

8. The admixture of claim 3 wherein the resin and bisphosphonate has a range of 1.5 to 2.0 free resin to 1.0 bisphosphonate w/w ratio.

9. The resinate of claim 5 wherein the bisphosphonate is near fully bound to the exchanger.

10. The resinate of claim 5 wherein the resin and bisphosphonates has a 1.5 to 1.0 w/w ratio.

11. The admixture of claim 5 wherein the resin and bisphosphonate bas a range of 1.5 to 2.0 free resin to 1.0 bisphosphonate w/w ratio.

12. The resinate of claim 6 wherein the bisphosphonate is present from about 25 mg to about 450 mg per tablet.

13. The resinate of claim 7 wherein the bisphosphonate is present from about 25 mg to about 450 mg per tablet.

14. The admixture of claim 8 wherein said resin contains about 25 mg to about 450 mg of bisphosphonate per tablet.

15. The resinate of claim 9 wherein the bisphosphonate is present from about 25 mg to about 450 mg per tablet.

16. The resinate of claim 10 wherein the bisphosphonate is present from about 25 mg to bout 450 mg per tablet.

17. The admixture of claim 11 wherein the bisphosphonate is present from about 25 mg to about 450 mg per tablet.

18. The resinate of claim 3 wherein a tablet for oral administration is prepared using pharmaceutical acceptable fillers and lubricants like lactose, silica, stearates, polyethylene glycol waxes, corn starch or other known exceipients.

19. The resin of claim 7 wherein the tablet for oral administration is prepared using pharmaceutical acceptable fillers and lubricants like lactose, silica, stearates, polyethylene glycol waxes, cornstarch or other known exceipents.

20. The admixture of claim 8 wherein a tablet for oral administration is prepared using pharmaceutical acceptable fillers and lubricants like lactose, silica, stearates, polyethylene glycol waxes, corn starch, or other known exceipents.

21. The resinate of claim 18 wherein the tablet is coating with a water soluble or hydrocolloid polymer like polyvinyl alcohol, cellulose esters or ethers, natural gums, polyvinyl pyrrolidione and the like to achieve additional drug delivery rates.

22. The resinate of claim 19 wherein the tablet is coated with a water soluble or hydrocolloid polymer like polyvinyl alcohol, cellulose esters or ethers, natural gums, polyvinyl pyrrolidione and the like to achieve additional drug delivery rates.

23. The admixture of claim 20 wherein the tablet is coated with a water-soluble or hydrocolloid polymer like polyvinyl alcohol, cellulose esters or eithers, natural gums, polyvinyl pyrrolidione and the like to achieve additional drug delivery rates.

24. The pharmaceutical composition or admixture as described in claim 1 whereby a second or third synergistic charged bioactive molecule is bound to the resin.

25. The pharmaceutical composition or admixture as described in claim 24 whereby the bioactive anionic molecule is an non-steroidal anti-inflammatory drug.

26. The pharmaceutical composition or admixture as described in claim 25 whereby the non-steroidal anti-inflammatory drug can be aspirin, ibuprofren, diclofenac, diflunisal, stodolac, fenoprofen, flurbiprofen, indomethacin, ketoprofen, kitofolac, mefenamic acid, naproxen, oxaprozin, piroxicam, salsalate, sulindac, tolmetin, and the like.

27. The pharmaceutical composition or admixture as described in claim 24 whereby the bioactive anionic molecule is a vitamin or pro-vitamin.

28. The pharmaceutical composition or admixture as described in claim 27 whereby the vitamin or pro-vitamin is vitamin C and/or vitamin D.

29. The pharmaceutical composition or admixture as described in claim 24 whereby the anionic molecule is a phosphate, polyphosphate or pyrophosphate.

30. The pharmaceutical composition or admixture as described in claim 24 whereby the anionic molecule is a chelating agent.

31. The pharmaceutical composition or admixture as described in claim 30 whereby the anionic molecule is an amino carboxylate or amino phosphonate.
